(19) United States
(12) Patent Application Publication
(10) Pub. No.: US 2006/0135644 A1
(43) Pub. Date: Jun. 22, 2006

(54) Dental polyalkenoate cement composition

(76) Inventors: Jurgen Engelbrecht, Elmshorn (DE); Ademola Olaseni Akinmade, Elmshorn (DE); Karl J. Gorlich, Elmshorn (DE)

Correspondence Address: FROMMER LAWRENCE & HAUG, 745 FIFTH AVENUE- 10TH FL., NEW YORK, NY, 10151, US

(21) Appl. No.: 11/221,253

(22) Filed: Sep. 7, 2005

(30) Foreign Application Priority Data
Sep. 7, 2004 (EP) EP 04 021 285.4

(51) Int. Cl. A61K 6/08; C08J 3/28

(52) U.S. Cl. 523/116; 523/300

(57) ABSTRACT

A dental polyalkenoate cement composition contains vitamin B or derivatives thereof as a “light-to-heat transformer”. Upon irradiation, the composition improves the clinical performance of a wide range of dental polyalkenoate cements products, such as: sealants; orthodontic and other specialty adhesives; and general restoratives.

The present invention relates to a dental polyalkenoate cement composition to be used for dental restoration or prevention of dental decay. The dental polyalkenoate cement composition according to the present invention especially contains at least one vitamin B compound like Riboflavin or their derivatives as a “light-to-heat transformer”.

In general, dental polyalkenoate cements are known per se cf. Acid-base cements, their biochemical and industrial applications, A. D. Wilson and J. W. Nicholson, Chemistry of Solid State Materials 3, Cambridge University Press, UK, 1993. They are used in a number of dental applications, including for example: as luting cements for the cementation of dental prostheses; crowns, inlays and bridges to teeth or for the cementation of orthodontic appliances to teeth; as filling cement for the restoration of dental cavities; as sealant cements for sealing pits and fissures in teeth; as luting cements for lining cavities; and as cement for core build-up.

The dental polyalkenoate cement belongs to a group of dental materials that exhibit good biocompatibility and chemical adhesive properties to tooth structure. In particular the dental glass-ionomer cement, a sub-set of polyalkenoate cements, is tooth-like, i.e. the translucency and pigmentation can be made to closely match that of natural tooth. Added to this is the well-known phenomenon of long-term, sustainable fluoride release from glass-ionomer cements. In fact, when the facts are considered and the properties of polyalkenoate cements are evaluated, they have the potential to be excellent dental materials for a wide range of dental remedies and preventive therapies. However, there are a number of limitations that prevent dental polyalkenoate cements from realizing their clinical potential. The features, benefits and limitations of polyalkenoate cements can be summarized in terms of their variants: i) the Classical water only-based polyalkenoate cement; and ii) the resin-modified glass-ionomer cement.

The “Classical” dental polyalkenoate cement comprises finely ground fluoro-aluminosilicate glass powder (or basic oxides), polyalkenoic acid(s) and water. The formation of the water-insoluble dental polyalkenoate cement from this water-based formulation takes place over a number of steps and over a period of time. In summary, in the presence of water, the polyalkenoic acid(s) attack the fluoro-aluminosilicate powder at their acid-susceptible sites and as a result liberate out ions (alkali metal ions, alkaline earth metal ions, and aluminum ions). These liberated metal ions undergo ionic bonding to alkenoic groups of the polyalkenoic acid(s) to form a cross linking structure. In this way the cement “salt” is gelled into a water-insoluble hard mass of material. The realization of this water-insoluble set polyalkenoate cement takes place rapidly over the initial several minutes from the start of the cement mix and then slower over several further hours and days. During this time the cement is susceptible to saliva attack.

A notable and suscessful attempt to improve the properties of the “Classical” glass-ionomer cement led to the development of resin-modified glass-ionomer cements [U.S. Pat. No. 4,872,936 October 1989]. In these cements, mainly hydrophilic resin monomers with pendant polymerisable acid groups have been introduced into the formulation of the “Classical” cement variant. In so doing, these polymerizable monomers can undergo light and/or chemically-induced polymerization in combination with the “Classical” glass-ionomer acid-base reaction. These resin-modified glass-ionomer cements are improvements on their “Classical” counterparts in terms of mechanical strengths, such as flexural strength and they also have better aesthetics, i.e. lower opacities.

Since dental glass-ionomer cements undergo time-dependent (acid-base ionomer) setting reactions, a certain length of time does elapse before the initial clinical set is attained. Of course, further clinical procedures are held up during this setting stage and within this time dental glass-ionomer cements, especially the Classical variant, are susceptible to saliva attack, as already pointed out. Saliva attack during initial setting is the process of metal ions' elution from the surface of dental glass-ionomer cement restorations due to the action of water from saliva. In this deleterious process, the water content of the cement increases and its metal ion-based cross-linking setting decreases. This results in more opaque restorations with lower surface hardness and, as a consequence, worse clinical wear and performance. In order to overcome this phenomenon, resin-based and other types of water-repellent varnishes are applied to the surfaces of freshly made glass-ionomer restorations. The efficacy of this practice has been reported to vary widely.

A number of attempts have been made to solve this Classical glass-ionomer problem of sluggish set leading to early susceptibility of external water attack. Notable amongst these attempts are 2 citations on the use of infrared energy to hasten the set of glass-ionomer cements [UK Patent No. GB 2 351 086 of January 1999] and [U.S. Pat. No. 6,264,472 of August 2000]. Another approach that has been proposed [UK Patent No. GB 2357773 A of October 2000], is the addition of coloring matter to dental glass-ionomer cements to reduce their L* values (from normally high values in commercial products) to below 60 and to irradiate such formulations with light in order to hasten their set. A disadvantage of this solution is that formulations with low L* values are dark and a deviation from the desirable tooth-like aesthetics of dental restorative products, including glass-ionomer cements. However, they are acceptable in dental applications with reduced visibility and where colour differentiations may be desirable.

The object of this invention is to present dental cements, including those with resin addition, which when irradiated with radiant energy set faster and possess the attendant benefits thereof. This is irrespective of L* values which in all our examples have remained above 60, due to the addition of Riboflavin or vitamin B or their derivatives.

This object is solved by a cement as disclosed in the claims. In particular, this object is solved by providing a dental cement composition containing a vitamin B compound like Riboflavin or derivatives thereof preferably in an amount from 0.001 to 10% by weight based on the whole cement composition, and more preferred from 0.01-1% by weight. The vitamin B compound and the riboflavin are able to absorb radiant energy within the range of the microwave, infrared, visible and/or ultraviolet spectrum, and to transform this energy into heat. Also the derivatives of the vitamin B compound are such derivatives which are able to absorb radiant energy within that range.

When radiant energy is shone on an inventive cement its temperature rises to the benefit of relatively disadvantageous polyalkenoate cement features, such as: slow speed of set; susceptibility to early water solubility; sluggish attainment of hardness; sluggish attainment of bond strength (cohesive strength), etc. Therefore, the above-mentioned inventive cements, upon radiant energy irradiation, improve the clinical performance of a wide range of dental polyalkenoate cements products, such as: sealants; orthodontic and other specialty adhesives; and general restoratives, especially in situations of low saliva management, e.g. in paedriatic and geriatric usage.

In this invention, the term “radiant energy” includes electromagnetic radiation (from any energy source) in the region of microwave, infrared, visible and ultraviolet regions of the electromagnetic spectrum. Preferable is the use of light having a wavelength from 300 to 800 nm, more preferably from 320 to 780 nm, as widely used in current dental remedy.

The temperature of the dental cement composition according to the present invention increases upon irradiation e.g. with light, and the time of initial setting is accelerated, whereby the time-dependent cement properties, such as susceptibility to attack by external water like saliva, hardness and cohesive strength development, etc. are improved.

The dental cement composition of the present inventon can also comprises a fluoro-aluminosilicate glass or basic oxides thereof. These compounds are preferably used as a powder which in a preferred embodiment—can be finally ground.

As the fluoro aluminosilicate glasses fluoro aluminosilicate glass powders and the like, which are generally used for dental glass-ionomer cements, are preferred. Of these, such fluoro aluminosilicate glass powders are preferred which have as the main components from 10 to 25% by weight of Al, from 5 to 30% by weight of Si, from 1 to 30% by weight of F, from 0 to 20% by weight of Sr, from 0 to 20% by weight of Ca, and from 0 to 10% by weight of alkali metal ions (e.g., Na, K etc.), based on the whole weight of the glass. Such glasses can be prepared by mixing raw materials containing these components and melting the mixture, and then cooling and pulverizing so as to have a mean particle size of from about 0.02 to 20 pm.

Of the basic oxide variant of the water-based polyalkenoate cement according to the present invention, preferable is the zinc oxide and deactivated zinc oxide/magnesium oxides of the zinc polycarboxylate cements. Preferred also are the newer zinc-based dental compositions [cf. European Patent No. EP 0883586, which is incorporated herein by reference]. Other less preferred basic oxide cements include those prepared from Be, Zn, Cu, Mg, Ca, Sr and Ba and combinations thereof.

The Vitamin B, especially Riboflavin, or their derivatives may be contained in any of the respective components of the cement: the fluoro-aluminosilicate glass powder; the aqueous solution of polyalkenoic acid(s); or in the components constituting the dental polyalkenoate cement composition in a powder, liquid or paste form. In addition, to the dental polyalkenoate cement compositions according to the present invention usual additives can be added like rheology modifiers, light absorbers like known ultraviolet light absorbers, plasticizers, antioxidants, bactericides, surfactants, etc., if desired. Unforeseen it could be observed that due to the content of energy absorbing substances the resin content of the cement could be lower than in commercial cements still achieving similar performance.

The dental cement composition according to the present invention can additionally comprise a resin. Resins, which can be added to dental cement compositions are known per se. The resin can, e.g. be present in the inventive composition as a bead, powder or liquid or combinations of these forms. It can be included in any of the components of the polyalkenoate powder, liquid or paste.

Polyacids as used in the present invention are especially polyalkenoic acids. Polyalkenoic acids and their use in dental cements are known per se. They are for example, described in Werner Kullmann, Atlas der Zahnerhaltung, Hanser Verlag, München (1990). The disclosure of this Atlas, and especially its disclosure concerning polyalkenoic acids is herewith enclosed by reference.

The dental polyalkenoate cement compositions according to the present invention will be hereunder described in more detail with reference to the following glass-ionomer cement Examples, but it is to be construed that the present invention should not be limited thereto.

### EXAMPLE 1

### The Effect of the Concentration of Riboflavin or Vitamin B or their Derivatives on the Temperature Rise in Classical Glass-Ionomer Cements

| | % Riboflavin 5′-mono | | |
|---|---|---|---|
| | phosphate, sodium salt | Temperature | Setting Time |
| Liquid | dihydrate | Rise, ° C. | [Sec] |
| Ref Liquid* | 0 | 0.6 | 240 |
| Liquid -1 | 0.0083 | 1.7 | 236 |
| Liquid -2 | 0.017 | 2.7 | 230 |
| Liquid -3 | 0.04 | 3.3 | 220 |
| Liquid -4 | 0.08 | 4.3 | 210 |

*Ref Liquid is the commercial product Vitro Molar Liquid from DFL, RJ Brasil (a classical glass-ionomer cement product). Liquids 1-4 were made by additions of Riboflavin to this product. The cements were formed by mixing the Vitro Molar powder with the liquid at a ratio of 1.4:1. The radiant energy source employed in all of the examples presented was Power Pac manufactured by American Medical Technologies (AMT), Corpus Cristi, USA.

It can be concluded:
1. Riboflavin 5′—mono phosphate, sodium salt dihydrate increased the setting exotherm of the cements and in so doing made them set faster.
2.

### EXAMPLE 2

### The Effect of Resin and Riboflavin or Vitamin B or Their Derivatives Inclusion on the Temperature Rise and Setting Properties of Glass-Ionomer Cements

| | Weight % of Constituents | | | | |
|---|---|---|---|---|---|
| | Liq- | Liq- | Liq- | Liq- | Liq- |
| Liquid Constituents | uid-5 | uid-6 | uid-7 | uid-8 | uid-9 |
| Riboflavin 5′-mono | 0.08 | 0.08 | 0.08 | 0.08 | 0.0 |
| phosphate, sodium salt | | | | | |
| dihydrate | | | | | |
| RM Lute liquid* | 0 | 3.3 | 6.6 | 12.5 | 12.5 |
| Vitro Molar Liquid | 99.9 | 96.7 | 93.4 | 87.5 | 87.5 |
| | 100 | 100 | 100 | 100 | 100 |
| Temperature rise ° C. | 4.3 | 4.8 | 10.3 | 12.2 | 0.6 |
| Setting Time without | 240 | 240 | 240 | 240 | 240 |
| light activation [sec] | | | | | |
| Setting Time with 40 s | 210 | 190 | 155 | 120 | 240 |
| radiant energy | | | | | |
| irradiation [sec] | | | | | |
| Decrease in Setting Time | 30 | 50 | 85 | 120 | 0 |
| with 40 s energy | | | | | |
| irradiation [sec] | | | | | |
| Decrease in Setting Time | 12.5 | 20.8 | 35.4 | 50.0 | 0.0 |
| with 40 s energy | | | | | |
| irradiation [%] | | | | | |

*RM Lute is a non light-cure resin-modified glass-ionomer commercial product of Strrouf Pharmaceuticals India. The cements were formed by mixing Vitro Molar powder with the liquids at powder: liquid of 1.4:1.

It can be concluded:
1. Riboflavin 5′—mono phosphate, sodium salt dihydrate has the effect of significantly raising the temperature of the polyalkenoate cements
2. In the absence of Riboflavin 5′—mono phosphate, sodium salt dihydrate, classical and resin-modified polyalkenoate cements have very low temperature rise when irradiated with the energy source.
3. The inclusion of Riboflavin 5′—mono phosphate, sodium salt dihydrate and especially in conjunction with resins has the effect of creating novel polyalkenoate cements with command-cure properties similar in some respect to those of traditional commercial resin-modified polyalkenoate cements.

Examples 1 and 2 have outlined the temperature rise feature and the benefit this has in enabling cements to set faster. Other potential benefits of these Riboflavin-containing products and the proposed curing method have been evaluated and are described following:

### EXAMPLE 3

### Increase in Early Surface Hardness of Potential Benefit for Fissure Sealing Applications and as an Alternative to the Varnishing of Dental Polyalkenoate Restorations

Sbore-D Hardness*

| | Without energy | With 40 s energy |
|---|---|---|
| Time interval | irradiation | irradiation |
| 5 mins from start of mix | 0 | 4 |
| 10 mins from start of mix | 20 | 28 |
| 25 mins from start of mix | 50 | 62 |
| 60 mins from start of mix | 80 | 80 |

The cement of this experiment was Liquid-8 mixed with the Vitro Molar powder, mixed at powder: liquid ratio of 1.4:1 and irradiated with radiant energy for 40 s.

### EXAMPLE 4

### Development of Early High Bond Strength to Un-Etched Enamel—of Potential Benefit in the Bonding of Orthodontic and Other Dental Appliances

| | Shear Bond Strength | | |
|---|---|---|---|
| | | Without light | With 40 s energy |
| Time Interval | | irradiation | irradiation |

| | | | |
|---|---|---|---|
| 30 mins ± 5 mins | 0 | MPa | 2.4 MPa |
| 3 hours ± 15 mins | 2.4 | MPa | 7.4 MPa |

Liquid 7 was mixed with Vitro Molar powder at powder: liquid ratio of 1.4:1 and applied to poly(acrylic acid) conditioned enamel and irradiated with radiant energy for 40 s.

### EXAMPLE 5

### The Reduction in Early Water Solubility of Glass-Ionomers with Riboflavin or its Derivates or Vitamin B

Total Dissolved Solids, mg/l

| | Without light | With 40 s light |
|---|---|---|
| Time Interval | irradiation | irradiation |
| 5.0 mins | 0.083 | 0.074 |
| 5.5 mins | 0.108 | 0.086 |
| 6.0 mins | 0.124 | 0.088 |
| 6.5 mins | 0.143 | 0.108 |
| 7.0 mins | 0.172 | 0.116 |
| 7.5 mins | 0.178 | 0.137 |
| 8.0 mins | 0.184 | 0.148 |
| 8.5 mins | 0.192 | 0.152 |

* Liquid 7 was mixed with Vitro Molar powder at powder: liquid ratio of 1.4:1 and 8.0 mg of the cement, 1 mm high was adhered to the bottom of glass bottle with 20 g of distilled water poured over it at t=4.0 minutes from the onset of mix. Energy irradiation was carried out for 40 s between t=3.0-4.0 minutes.

1. A dental polyalkenoate cement composition comprising at least one Vitamin B compound or derivative thereof which are able to absorb radiant energy within the range of the microwave, infrared, visible and/or ultraviolet spectrum, and to transform this energy into heat.

2. The dental cement composition according to claim 1 wherein the vitamin B compound and/or its derivative are contained, based on the whole composition, in an amount from 0.001% to 10% by weight and preferably from 0.01% to 1% by weight.

3. The dental cement composition according to claim 1 wherein the radiant energy preferably has a wavelength from 300-800 nm.

4. The dental cement composition according to claim 1 additionally comprising at least one fluoro-aluminosilicate glass powder or basic oxides thereof, polyacid(s) and water.

5. The dental cement composition according to claim 1 additionally comprising at least one resin.

6. The dental cement composition according to claim 1 additionally comprising usual additives like rheology modifiers, plasticizers, light absorbers, especially absorbers of ultraviolet light, antioxidants, bactericides and/or surfactants.

7. The dental cement composition according to claim 5 wherein the resin is a mono- or di- or tri- or multi-methacrylate and optionally includes polymerization activators, initiators and/or other redox reaction systems.

8. The dental cement composition according to claim 5 wherein the resin content is lower than 20 weight percent, based on the whole composition.

9. The dental cement composition according to claim 5 wherein the resin content is lower than 10 weight percent, based on the whole composition.

10. The dental cement composition according to claim 5 wherein the resin content is lower than 5 weight percent, based on the whole composition.
